(19) United States
(12) Patent Application Publication
(10) Pub. No.: US 2026/0315429 A1
(43) Pub. Date: Oct. 15, 2026

(54) TREATMENT COMPOSITION FOR MILD COGNITIVE IMPAIRMENT AND DEMENTIA

(71) Applicant: Zyvran Pharmaceutical Inc., d/b/a Zyvran Pharmaceuticals, d/b/a Zyvran Pharma, Los Angeles, CA (US)

(72) Inventors: Taghogho Agarin, San Jose, CA (US); William Van Horn, Mount Pleasant, SC (US); Carolyn Godman, San Diego, CA (US)

(73) Assignee: Zyvran Pharmaceutical Inc., d/b/a Zyvran Pharmaceuticals, d/b/a Zyvran Pharma, Los Angeles, CA (US)

(21) Appl. No.: 19/708,193

(22) Filed: Jun. 15, 2026

Related U.S. Application Data
(62) Division of application No. 18/238,229, filed on Aug. 25, 2023.
(60) Provisional application No. 63/405,345, filed on Sep. 9, 2022.

(51) Int. Cl. A61K 31/27; A61K 31/137

(52) U.S. Cl. CPC A61K 31/27; A61K 31/137

(57) ABSTRACT

A method for treating mild cognitive impairment and dementia is provided. The method comprises administering to a patient in need a therapeutically effective dosage of a composition comprising a cholinesterase inhibitor and a serotonin and norepinephrine reuptake inhibitor (SNRI). The composition may further comprise an N-methyl-D-aspartate (NMDA) receptor modulator. In embodiments, the cholinesterase inhibitor is selected from rivastigmine, donepezil, or galantamine, and the SNRI is selected from duloxetine, venlafaxine, or desvenlafaxine. In certain embodiments, the cholinesterase inhibitor is rivastigmine and the SNRI is venlafaxine, desvenlafaxine, or duloxetine. In further embodiments, the NMDA receptor modulator is memantine hydrochloride. The therapeutically effective dosage may comprise about 1 mg to about 12 mg of the cholinesterase inhibitor and about 10 mg to about 400 mg of the SNRI, and optionally about 1 mg to about 45 mg of the NMDA receptor modulator.

### RELATEDAPPLICATION

This application is a divisional of U.S. Application No. 18/238,229 filed on Aug. 25, 2023, which claims priority to provisional patent application U.S. Serial No. 63/405,345 filed on Sept. 9, 2022, the entire contents of which is herein incorporated by reference.

### BACKGROUND

The embodiments described herein relate generally to treatment compositions and, more particularly, to a treatment composition for treating dementia, such as Parkinson's disease, Dementia with Lewy Bodies, and Alzheimer's Dementia.

Alzheimer's disease is the most common type of dementia. It causes a progressive deterioration causing the brain to have reduced volume and causing brain cells to die. It affects memory, behavior, and social skills, which impacts the ability of those affected to function independently. Affected patients develop memory problems, such as forgetting and difficulty conducting everyday tasks. Alzheimer's dementia affect roughly 6 million people in the United States, many of which are 65 years and older.

Dementia with Lewy Bodies (DLB) is a common degenerative dementia and becomes more common with age, accounting for approximately 5% of all dementia cases in older populations. It is a neurodegenerative disorder similar to Parkinson's disease. The essential component of a diagnosis of DLB is dementia, a loss of cognitive functioning that is sever enough to interfere with a person's daily activities and functioning. DLB is caused by the accumulation of aggregated forms of the protein a-synuclein in neuronal and non-neuronal cells in the brain. The core clinical features of DLB are fluctuating cognitive impairment, visual hallucinations, rapid eye movement sleep disorder, and spontaneous extrapyramidal motor problems, such as bradykinesia, rest tremor, and rigidity. Other clinical features include severe sensitivity to antipsychotic medications, severe autonomic dysfunction, postural instability, hypersomnia, anxiety, depression, and other types of hallucinations or delusions. There are also specific biomarkers associated with DLB. Current treatments for DLB include taking multiple separate medications, wherein each of the separate medications targets a different symptom of DLB. Taking multiple mediations, however, is not realistic for this population, as this requires the administration of multiple medications simultaneously and requires a patient to keep track of which medications have been taken, at what dosages, and when. As such, there is much room for error in the administration of the medications to treat DLB.

Therefore, what is needed is a method and composition for treating Dementia with Lewy Bodies, Parkinson's Dementia, and Alzheimer's Dementia, wherein the composition is a single administration treatment and is intended to provide symptomatic relief of behavioral symptoms.

### SUMMARY

Some embodiments of the present disclosure include a composition for the treatment of mild cognitive impairment and dementia. The composition may include a cholinesterase inhibitor and a serotonin and norepinephrine reuptake inhibitor (SNRI). The composition may further include an N-methyl-D-aspartate receptor modulator. The cholinesterase inhibitor may be rivastigmine, donepezil, or galantamine. The SNRI may be duloxetine, venlafaxine, or desvenlafaxine. The N-methyl-D-aspartate receptor modulator may be memantine hydrochloride. A method for treating mild cognitive impairment and dementia may include administering, to a patient in need, a therapeutically effective dosage of the composition.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

The composition of the present disclosure may be used as a single administration treatment for treating mild cognitive impairment and dementia and may comprise the following elements. This list of possible constituent elements is intended to be exemplary only, and it is not intended that this list be used to limit the composition of the present application to just these elements. Persons having ordinary skill in the art relevant to the present disclosure may understand there to be equivalent elements that may be substituted within the present disclosure without changing the essential function or operation of the composition.

The various elements of the present disclosure may be related in the following exemplary fashion. It is not intended to limit the scope or nature of the relationships between the various elements, and the following examples are presented as illustrative examples only.

By way of example, some embodiments of the present disclosure include a method and composition for the treatment of mild cognitive impairment (MCI) and dementia, wherein the method comprises administering, to a patient in need, a therapeutically effective dosage of a single administration composition comprising a cholinesterase inhibitor and a serotonin and norepinephrine reuptake inhibitor (SNRI). In some embodiments, such as those used to treat severe cognitive impairment and dementia, the composition may further comprise a N-methyl-D-aspartate receptor modulator. In embodiments, the single administration composition may be provided in pill form, wherein only a single pill needs to be administered to a patient to provide a therapeutically effective dosage. As used herein, a therapeutically effective dosage may comprise an amount of the composition that produces a sufficient therapeutic response.

In embodiments, the cholinesterase inhibitor may be, for example, rivastigmine, donepezil, or galantamine, and the SNRI may be duloxetine, venlafaxine, or desvenlafaxine. In a particular embodiment, the cholinesterase inhibitor may be rivastigmine and the SNRI may be a member selected from the group consisting of venlafaxine and desvenlafaxine. When included, the N-methyl-D-aspartate receptor modulator may be memantine hydrochloride.

In embodiments, the composition may comprise, for example, about 0.25 weight % (wt.%) to about 55 wt.% cholinesterase inhibitor and about 45 wt.% to about 99.75 wt.% SNRI. When included, the composition may comprise from about 3 wt. % to about 5 wt. % of the N-methyl-D-aspartate receptor modulator. As mentioned above, the single administration composition may be provided in a therapeutically effective dosage. In embodiments, the therapeutically effective dosage of the composition may comprise from about 1 mg to about 12 mg cholinesterase inhibitor and about 10 mg to about 400 mg SNRI. In some embodiments, the cholinesterase inhibitor may be rivastigmine and the SNRI may be venlafaxine, wherein the therapeutically effective dosage may comprise from about 1 mg to about 12 mg rivastigmine and about 10 mg to about 300 mg venlafaxine. In an alternate embodiments, the cholinesterase inhibitor may be rivastigmine and the SNRI may be desvenlafaxine, wherein the therapeutically effective dosage may comprise from about 1 mg to about 12 mg rivastigmine and from about 25 mg to about 400 mg desvenlafaxine. When included, the therapeutically effective dosage may comprise about 1 mg to about 45 mg N-methyl-D-aspartate receptor modulator, such as memantine hydrochloride.

In embodiments, the combination of rivastigmine and venlafaxine or desvenlafaxine in a single pill composition may provide relief of symptoms in dementia with Lewy bodies as well as other forms of dementia. For the treatment of severe cases of dementia, memantine hydrochloride may be added to the single pill composition. In fact, the combination of ingredients into a single pill may result in a synergistic combination, wherein the combination works better than administering the ingredients independently to the patient in need. Particularly, the cholinesterase portion of the composition may improve cognition, whereas the SNRI portion may improve daytime alertness and behavioral disinhibition. Together, the ingredients create the desired neurochemical balance to achieve good functioning. More specifically, the treatment may improve behavior and alertness within days of starting administration thereof. It may also improve symptoms of behavioral agitation, promote daytime wakefulness, and improve behavioral disinhibition of Alzheimer's disease.

While the above descriptions indicates that the composition may be administered as a single pill, the composition is not limited to such administration. Rather, the composition may be administered in tablets, capsules, oral disintegrating tablets, oral disintegrating strips, oral dispersible films, liquid solutions, extended release preparations, controlled release forms, lozenges, films, nasal sprays, patches or topical cream applications. Moreover, the composition may be provided in a therapeutically effective dosage, which may be administered on-demand or in set, pre-determined periods of time. Furthermore, the composition may be provided in a single dosage form or in a split dosage form multiple times a day.

The above-described embodiments of the invention are presented for purposes of illustration and not of limitation. While these embodiments of the invention have been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claims.

What is claimed is:

1. A method for treating mild cognitive impairment and dementia, the method
comprising:
administering a therapeutically effective dosage of a composition to a patient in need,
wherein the composition comprises:
a cholinesterase inhibitor; and
a serotonin and norepinephrine reuptake inhibitor (SNRI).

2. The method of claim 1, wherein the composition further comprises an N-methyl-
D-aspartate receptor modulator.

3. The method of claim 1, wherein a therapeutically effective dosage of the
composition comprises:
about 1 mg to about 12 mg cholinesterase inhibitor; and
about 10 mg to about 400 mg SNRI.

4. The method of claim 1, wherein:
the cholinesterase inhibitor is a member selected from the group consisting of
rivastigmine, donepezil, and galantamine; and
the SNRI is a member selected from the group consisting of duloxetine,
venlafaxine, and desvenlafaxine.

5. The method of claim 4, wherein:
the cholinesterase inhibitor is rivastigmine; and
the SNRI is a member selected from the group consisting of venlafaxine and
desvenlafaxine.

6. The method of claim 4, wherein the SNRI is duloxetine.

7. The method of claim 4, wherein:
the cholinesterase inhibitor is rivastigmine; and
the SNRI is duloxetine.

8. The method of claim 2, wherein:
the cholinesterase inhibitor is rivastigmine;
the SNRI is duloxetine; and
the N-methyl-D-aspartate receptor modulator is memantine hydrochloride.

9. The method of claim 2, wherein the therapeutically effective dosage of the composition further comprises:
about 1 mg to about 45 mg of the N-methyl-D-aspartate receptor modulator.

10. The method of claim 1, wherein the composition comprises:
10 about 0.25 wt. % to about 55 wt. % cholinesterase inhibitor; and
about 45 wt. % to about 99.75 wt. % SNRI.
